(19) United States
(12) Patent Application Publication
(10) Pub. No.: US 2026/0344861 A1
(43) Pub. Date: Nov. 5, 2026

(54) HAIRCARE APPLIANCE WITH A SENSOR-EMITTER ARRANGEMENT

(71) Applicant: Dyson Technology Limited, Malmesbury, Wiltshire (GB)

(72) Inventors: Thomas Alexander CARLYLE, Chippenham (GB); Nathan Haruna Waters IMHASLY, Bath (GB); Adam BRANN, Bristol (GB)

(21) Appl. No.: 19/119,977

(22) PCT Filed: Oct. 5, 2023
(86) PCT No.: PCT/IB2023/060006

§ 371 Date: Apr. 10, 2025

(30) Foreign Application Priority Data
Oct. 11, 2022 (GB) 2214960.3

(51) Int. Cl. A45D 20/12; A45D 44/00

(52) U.S. Cl. CPC A45D 20/12; A45D 2044/007

(57) ABSTRACT

A haircare appliance has a main body with an annular outlet that expels an airflow in the form of an annular column. The haircare appliance also has a sensor-emitter arrangement that is located radially inwardly of the annular outlet. The sensor-emitter arrangement has at least one of a sensor and an emitter for sensing an attachment attached to the main body, or to sense a property of, or to irradiate hair that is proximate to the haircare appliance.

### FIELD OF THE INVENTION

The present invention relates to a haircare appliance comprising a sensor-emitter arrangement.

### BACKGROUND OF THE INVENTION

A haircare appliance may incorporate sensors or emitters. For example, a sensor may sense hair or an attachment that is attached to a main body of the haircare appliance. However, incorporating sensors or emitters into a haircare appliance can present challenges.

### SUMMARY OF THE INVENTION

According to a first aspect of the present invention, there is provided a haircare appliance comprising: a main body comprising an annular outlet configured to expel an airflow in the form of an annular column; and a sensor-emitter arrangement located radially inwardly of the annular outlet, wherein the sensor-emitter arrangement comprises at least one of a sensor and an emitter configured to sense an attachment attached to the main body, or to sense a property of or to irradiate hair proximate the haircare appliance.

Locating the sensor-emitter arrangement radially inwardly of the annular outlet may allow the sensor-emitter to be aligned with the expelled airflow path or a target of the expelled airflow path (such as a user's head or hair) without disrupting the expelled airflow itself. Accordingly, accurate sensing and/or accurately directed irradiation may be provided without negatively affecting or impacting the expelled airflow. Alternatively or additionally, locating the sensor-emitter arrangement radially inwardly of the annular outlet may allow for the sensor-emitter arrangement to be located away from a heated airflow or other components of the haircare appliance which may otherwise interfere with the sensor or emitter. This may, in turn, reduce heat or other protection that may otherwise be needed for the sensors or emitters and/or allow for sensors or emitters that may be damaged or otherwise negatively affected by heat or other interference to nonetheless be used. Alternatively or additionally, locating the sensor-emitter arrangement radially inwardly of the annular outlet may allow the sensor or emitter to be incorporated into the haircare appliance without requiring significant modification of the packaging of the haircare appliance and/or increasing the overall size of the hair care appliance, which may be cost effective.

The haircare appliance may comprise a barrel section that comprises the annular outlet, the barrel section may comprise a bore, and the sensor-emitter arrangement may be located in the bore.

Locating the sensor-emitter arrangement in the bore may allow for the sensor-emitter arrangement to be relatively physically isolated from other components located in the barrel section, such as a heater, which may reduce heat transfer and/or other interference from those components. Alternatively or additionally, this may allow a clear line of sight from the senor-emitter arrangement out of the haircare appliance. This may, for example, be useful where the sensor senses an attachment or a property of hair using a light-based sensing method and/or where the emitter irradiates hair proximate to the haircare appliance.

The main body may surround or house the sensor-emitter arrangement. This may allow for the main body to physically protect the sensor-emitter arrangement, which may provide for a robust haircare appliance.

The sensor-emitter arrangement may be located on a central axis of the annular outlet. This may allow for an ideal alignment of the sensor/emitter arrangement with the expelled airflow. Alternatively or additionally, this may allow the sensor-emitter arrangement to be equidistant from a heated airflow, which may reduce the extent to which hotspots may develop on the sensor-emitter arrangement.

The sensor-emitter arrangement may comprise at least one of a light-based sensor and a light-based emitter. For example, the sensor-emitter arrangement may comprise one or more of a time-of-flight sensor, an imaging device such as a camera, a spectroscopy and/or reflectometry device, and a light-based hair, skin or scalp treatment device such as a Red Light Therapy emitter, Low-Level Laser Therapy emitter, or an Ultraviolet C (UVC) emitter. Being located radially inwardly of the annular outlet allows emitted and/or received light to be aligned with the expelled airflow, which may allow for more accurate sensing of and/or emitting onto the target of the expelled airflow.

The light-based sensor may be configured to sense light in a first direction or the light-based emitter may be configured to emit light in the first direction, and the first direction may be parallel to the central axis of the annular outlet. This may help ensure that the emitted and/or received light is aligned with the expelled airflow, which may allow for more accurate sensing of and/or emitting onto the target of the expelled airflow.

The sensor-emitter arrangement may comprise a proximity sensor or a magnetometer. Locating a proximity sensor radially inwardly of the annular outlet may allow for an alignment of the target of the proximity sensor (such as a user's head or hair) with the airflow target. This may, in turn, allow for an accurate and/or reliable determination of a proximity, e.g. a distance to a user's head or hair, and hence e.g. allow for improved operation of the haircare appliance. For example, the proximity sensor may comprise a time-of-flight sensor. The magnetometer may allow for magnetic attachments to be discriminated between without providing a magnetometer at an attachment interface of the main body and the attachment, which may have tight packaging constraints. Alternatively or additionally, placing the magnetometer radially inwardly of the annular outlet may reduce interference from electrical components of the haircare appliance such as the heater.

The sensor-emitter arrangement may comprise a visual indicator device configured to indicate a current operating mode of the haircare appliance to a user. For example, the visual indicator device may be an LED. For example, the visual indicator device may indicate an operating mode of the sensor-emitter arrangement, for example whether or which sensing or emitting mode the sensor-emitter arrangement is currently operating in. The sensor-emitter arrangement comprising the visual indicator device may allow for an operating mode to be communicated to the user without significantly altering a display configuration of main body of the haircare appliance itself, which may be cost effective.

The main body may comprise an annular heater over which the airflow passes before being expelled, and the sensor-emitter arrangement may be located radially inwardly of the annular heater. This may allow for the temperatures to which the sensor or emitter is exposed to be reduced, which may in turn reduce heat protection that may otherwise be needed for the sensor or emitter and/or allow for the sensor or emitter that may be otherwise be damaged by heat or suffer from other interference to nonetheless be used.

The sensor-emitter arrangement may comprise a capsule housing the at least one of a sensor and an emitter, and the capsule may be connected to the main body by a support member. This arrangement may allow for minimal heat to be transferred from main body to the capsule. For example, the capsule may be connected to the main body by a single rib, which may minimise the heat transferred from the main body to the capsule.

The capsule may be elongate in a direction parallel that of the expelled airflow. This may allow to reduce interference with a secondary airflow that may be drawn through haircare appliance (e.g. through a bore in which the senor-emitter arrangement is located) when air is expelled from the annular outlet. This may allow for improved operation of the haircare appliance.

The haircare appliance may comprise a control module configured to control operation of the haircare appliance in dependence on an output of the sensor-emitted arrangement. This may allow for the haircare appliance to be automatically controlled. Operation of the haircare appliance may comprise controlling a heater and/or an airflow generator of the haircare appliance. For example, where the sensor senses a certain attachment, the control module may control the haircare appliance to operate (e.g. the heater and/or the airflow generator) with settings according to the certain attachment. For example, attachments may comprise a diffuser and a concentrator. The diffuser may provide better styling results when a relatively low airflow rate is used, whereas the concentrator may provide better styling results when a relatively high airflow rate is used.

The haircare appliance may comprise an attachment attachable to the main body to receive the expelled airflow from the annular outlet, and the sensor-emitter arrangement may be configured to sense the attachment, or to sense a property of or to irradiate hair proximate to the attachment, when the attachment is attached to the main body. This may allow for the sensor-emitter arrangement to function in the case where an attachment is attached to the main body. This may improve the flexibility with which the haircare appliance can be used.

The attachment may comprise a transport path along which emissions pass to or from the sensor-emitter arrangement. This may allow for emissions, such as electromagnetic radiation, to pass from the sensor-emitter arrangement to the head or hair of the user (or vice versa) when the attachment is attached to the main body. This may allow for flexibility in the functioning of the haircare appliance whilst still allowing for the hair sensing and/or irradiating functionality of the haircare appliance.

The sensor-emitter arrangement may comprise a magnetometer, the attachment may be one of a plurality of magnetic attachments, and the haircare appliance may comprise a control module configured to determine which of the plurality of magnetic attachments is attached to the main body based on data output by the magnetometer. This may allow for the control module to determine which of a plurality of magnetic attachments is attached to the main body. For example, the different magnetic attachments may differ in the magnetic field produced at the magnetometer when the magnetic attachment is attached to the main body. This may be sensed by the magnetometer, and the control module may identify a given one of the magnetic attachments from among the plurality based on the output from the magnetometer indicating a given magnetic field at the magnetometer. The magnetometer being located radially inwardly of the annular outlet may allow for the attachment to be determined remotely from the attachment interface (i.e. the region of the main body where attachments attach to the main body), which may have tight packaging constraints.

The sensor-emitter arrangement may comprise a time-of-flight (TOF) sensor, the attachment may be one of a plurality of attachments, and the haircare appliance may comprise a control module configured to determine which of the plurality of magnetic attachments is attached to the main body based on data output by the time-of-flight sensor. This may allow the control module to determine which of a plurality of attachments is attached to the main body. The TOF sensor being located radially inwardly of the annular outlet may allow for the attachment to be determined remotely from the attachment interface, which may have tight packaging constraints. Alternatively or additionally, the TOF sensor may be used both to determine which attachment is attached to the main unit and to sense a proximity of hair to the attachment. Using one sensor for two functions may provide a cost-effective arrangement, for example as compared to providing separate sensors for the separate functions.

The sensor-emitter arrangement may be located on the central axis of the annular outlet and the attachment, when attached to the main body, may be rotatable relative to the main body about the central axis. This may allow that the sensor-emitter arrangement is aligned with a rotational axis of the attachment. This may allow the sensor-emitter arrangement to function regardless of the rotational position of the attachment. This may improve flexibility of use of the attachment. For example, this may allow that the sensor-emitter arrangement may be aligned with an emissions transport path of the attachment regardless of the rotational position of the attachment and/or that the magnetometer can identify a magnetic attachment regardless of the rotational position of the magnetic attachment. The attachment being rotatable relative to the main body about the central axis may allow for improved functionality and/or ease of use of the haircare appliance.

According to a second aspect of the present invention, there is provided a haircare appliance comprising: a main body comprising a barrel section having a central bore and an outlet located at an end of the barrel section to expel an airflow in the form of an annular column; and a sensor-emitter arrangement located within the bore, the sensor-emitter arrangement comprising at least one of a sensor and an emitter. Similar to as described above, the sensor-emitter arrangement being located within the bore may allow the sensor-emitter to be aligned with the expelled airflow path or a target of the expelled airflow path (such as a user's head or hair) without disrupting the expelled airflow itself. Accordingly, accurate sensing and/or accurately directed irradiation may be provided without negatively affecting or impacting the expelled airflow. Alternatively or additionally, locating the sensor-emitter arrangement within the bore may allow for the sensor-emitter arrangement to be relatively physically isolated from other components located in the barrel section, such as a heater, which may reduce heat transfer and/or other interference from those components. Alternatively or additionally, locating the sensor-emitter arrangement within the bore may allow the sensor or emitter to be incorporated into the haircare appliance without requiring significant modification of the packaging of the haircare appliance and/or increasing the overall size of the hair care appliance, which may be cost effective. Alternatively or additionally, locating the sensor-emitter arrangement within the bore may allow a clear line of sight from the sensor-emitter arrangement out of the haircare appliance. This may, for example, be useful where the sensor senses an attachment or a property of hair using a light-based sensing method and/or where the emitter irradiates hair proximate to the haircare appliance.

### BRIEF DESCRIPTION OF THE DRAWINGS

Further features and advantages will now be described, by way of example only, with reference to the accompanying drawings of which:

FIG. 1 is a schematic diagram illustrating a perspective view of a haircare appliance according to an example;

FIG. 2 is a schematic diagram illustrating a rear view of the example haircare appliance;

FIG. 3 is a schematic diagram illustrating a partial front view of the example haircare appliance;

FIG. 4 is a schematic diagram illustrating a side cross sectional view of the example haircare appliance;

FIG. 5 is a schematic diagram illustrating a side cross sectional view of the example haircare appliance including an attachments; and

FIG. 6 is a schematic diagram illustrating a perspective view of the example haircare appliance with a plurality of attachments.

As used herein, like reference signs denote like features. The axes x, y, z indicated in each Figure correspond amongst the Figures.

### DETAILED DESCRIPTION OF THE INVENTION

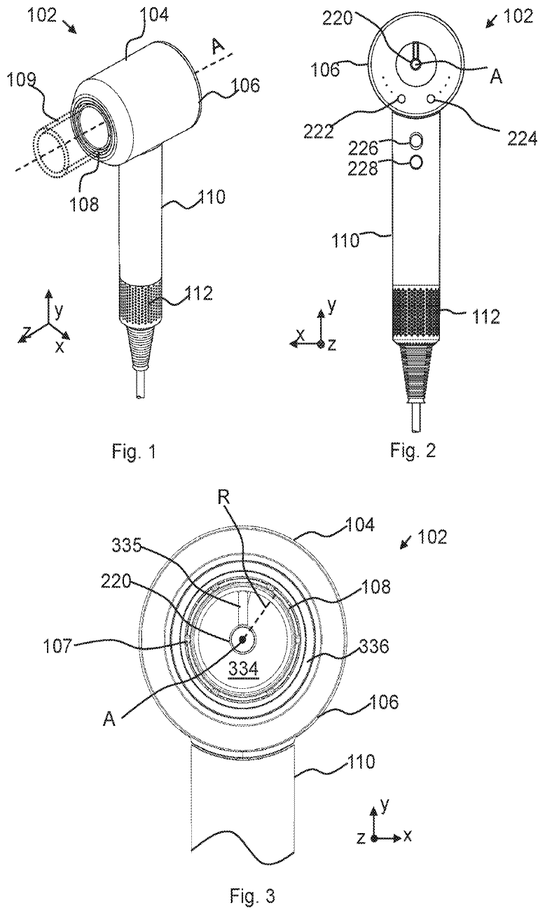

Referring to FIGS. 1 to 6, there is illustrated a haircare appliance according 102 to an example. In broad overview, the haircare appliance 102 comprises a main body 104 comprising an annular outlet 108 configured to expel an airflow 109 in the form of an annular column 109. The haircare appliance 102 also comprises a sensor-emitter arrangement 220 located radially inwardly of the annular outlet 108. The sensor-emitter arrangement 220 comprises at least one of a sensor 361, 363 and an emitter 361 configured to sense an attachment 114, 116 attached to the main body 104, or to sense a property of or to irradiate hair 404 proximate the haircare appliance 102.

Locating the sensor-emitter arrangement 220 radially inwardly of the annular outlet 108 may allow the sensor 361, 363 and/or emitter 361 to be aligned with the expelled airflow 109 path or a target of the expelled airflow 109 path (such as a user's head or hair 404) without disrupting the expelled airflow 109 itself. Accordingly, accurate sensing and/or accurately directed irradiation may be provided without negatively affecting or impacting the expelled airflow 109. As is known per se in hairdryers, the expelled airflow 109 may be heated. Alternatively or additionally therefore, locating the sensor-emitter arrangement 220 radially inwardly of the annular outlet 108 may allow for the sensor-emitter arrangement 220 to be located away from a heated airflow 109 or other components such as a heater 330 of the haircare appliance 102 which may otherwise interfere with the sensor 361, 363 or emitter 361. This may, in turn, reduce heat protection or other protection that may otherwise be needed for the sensors 361, 363 or emitters 361 and/or allow for sensors 361, 363 or emitters 361 that may be damaged or otherwise negatively affected by heat or other interference to nonetheless be used in the senso-emitter arrangement 220. Alternatively or additionally, locating the sensor-emitter arrangement 220 radially inwardly of the annular outlet 108 may allow the sensor or emitter to be incorporated into the haircare appliance 102 without requiring significant modification of the packaging of the haircare appliance 102 and/or increasing the overall size of the hair care appliance 102, which may be cost effective.

In the specific example illustrated in FIGS. 1 to 6, the haircare appliance is a hairdryer. The main body 104 of the haircare appliance 102 comprises a handle section 110 and a barrel section 106. The handle section 110 is generally cylindrical in shape and comprises a housing 337 that houses an airflow generator 332. The housing 337 comprises an inlet 112 through which an airflow is drawn into the handle section 110 by the airflow generator 332, and an outlet 350 through which the airflow is discharged into the barrel section 106. The airflow generator 332 may comprise, for example, a fan driven by an electric motor.

The barrel section 106 is likewise generally cylindrical in shape, but is shorter in length and wider in diameter than the handle section 112. The barrel section 106 is attached to an end of the handle section 112 and is oriented such that the longitudinal axes of the handle section 112 and the barrel section 106 are orthogonal. As a result, the shape of the main unit 104 resembles a gavel or mallet.

The barrel section 106 comprises a housing 301 that houses a heater 330 and a control module 315. The housing 301 comprises an outer wall 301a and an inner wall 301b that are generally concentric and define a chamber, specifically an annular chamber, within which the heater 330 and the control module 315 are housed. The housing 301 comprises an inlet 351 through which airflow from the handle section 112 enters the chamber, and the annular outlet 108 at an end of the barrel section 104 through which the airflow is discharged. The heater 330 is located between the inlet 351 and the outlet 108 and, when powered, heats the airflow. The inner wall 301b defines a bore 334 that extends through the centre of the barrel section 106.

As best seen in FIG. 2, the main body 104 further comprises user controls 222, 224, 226, 228. The user controls 222, 224, 226, 228 are provided on both the handle section 110 and the barrel section 106, and comprise a first button 226 or slider to power on and off the appliance 102, a second button 228 to momentarily power off the heater 330 such that the appliance 102 delivers a cold shot of air, a third button 222 to control the flow rate of the airflow, and a fourth button 224 to control the temperature of the airflow. The control module 315 may control electric components 332, 330, 361 in response to user controls. For example, in response to inputs from the user controls 222, 224, 226, 228, the control module 315 may power on and off the airflow generator 332 and/or the heater 330. Additionally, the control module 315 may control the power or speed of the airflow generator 332 in order to vary the flow rate of the airflow. For example, repeatedly pressing the third button 228 may cause the control module 315 to cycle through different flow rates (e.g., low, medium and high). Similarly, the control module 315 may control the power of the heater 330 in order to vary the temperature of the airflow. For example, repeatedly pressing the fourth button 224 may cause the control module 315 to cycle through different temperature settings (e.g., cold, warm, hot).

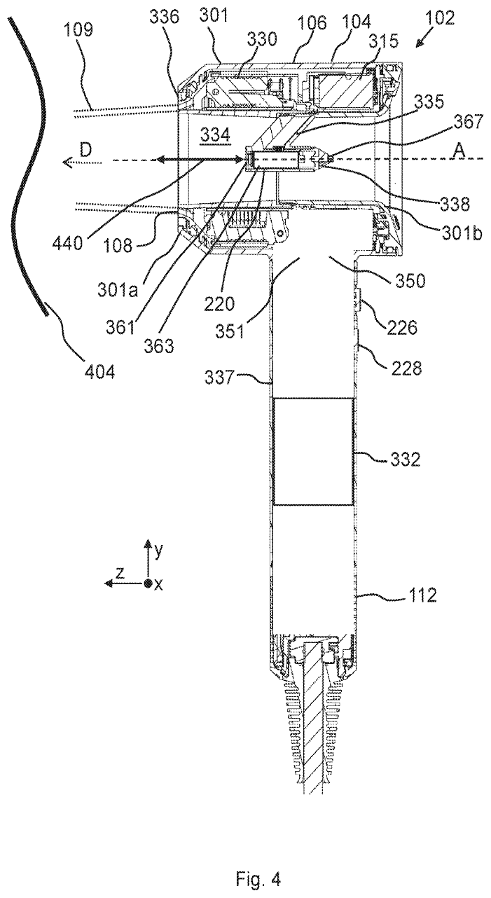

As mentioned, the outlet 108 is annular and is configured to expel the airflow 109 in the form of an annular column 109. The annular outlet 108 may be continuous or discontinuous. For example, as best seen in FIG. 3, the haircare appliance 102 may comprise thin ribs 107 connecting the outer wall 301a and the inner wall 301b and which cross the annular outlet 108. In this case the annular outlet 108 may be described as discontinuous, but nonetheless is an annular outlet configured to expel an airflow 109 in the form of an annular column 109. In examples where there are no such thin ribs 107, the annular outlet 108 may be described as continuous. The annular column 109 of expelled airflow may take different forms. For example, in some examples, the annular column 109 may take the general form of a hollow cylinder. However, other forms may be used. For example, as best seen in FIG. 4, the sides of the annular column 109 may be angled towards a central axis A of the barrel section 106 of the main body 104. In this case, the annular column 109 may be said to take the form of a hollow frustrum of a cone. In the illustrated example, the annular outlet 108 and accordingly the annular column 109 of expelled airflow are circular. This may provide, for example, for an even airflow and hence even drying as well as for compatibility with and flexibility of use of attachments 114, 116. However, in some examples (not illustrated) the annular outlet 108 and annular column 109 need not necessarily be circular and for example may take the form of other shapes, such as any polygonal annular outlet (not shown) and/or any polygonal annular column (not shown). Accordingly, it will be appreciated that the outlet 108 may be annular in the sense that the outlet is in the form of a ring or loop (of some shape) with a central hole and/or part where the outlet 108 is not provided (in the illustrated example the central hole and/or part where the outlet 108 is not provided is formed of the inner wall 310b and the bore 334). Indeed, as per the illustrated example, the airflow generated by and within the haircare appliance 102 and which is to be expelled from the haircare appliance 102 may not be provided to or in this central hole or part 310b, 334. Similarly, the column of air 109 that is expelled may be annular in the sense that the column of expelled air 109 has a central hole where the expelled airflow 109 is not present. Indeed, as per the illustrated example, the airflow generated by and within the haircare appliance 102 and which is expelled from the haircare appliance 102 at the outlet 108 is not present in this central hole. In any case, locating the sensor-emitter arrangement 220 radially inwardly of the annular outlet 108 may allow for the sensor-emitter arrangement 220 to be aligned with the expelled airflow 109 but without interfering with (e.g. being in the path of) the expelled airflow 109. Indeed, as per the illustrated example, the sensor-emitter arrangement 220 is not in the path of the airflow generated by and within the haircare appliance 102 and which is to be expelled from the haircare appliance from the annular outlet 108. Accordingly, the sensor-emitter arrangement 220 does not interfere with the generated airflow, which may provide for efficient operation of the haircare appliance 102.

As mentioned, the sensor-emitter arrangement 220 is located radially inwardly of the annular outlet 108. Specifically, in the illustrated example, the sensor-emitter arrangement 220 is located in the bore 334. More specifically, in the illustrated example, the sensor-emitter arrangement 220 is located on the central axis A of the annular outlet 108 (which in the illustrated example is the same as the central longitudinal axis A of the barrel section 106 of the main body 104). Locating the sensor-emitter arrangement 220 on the central axis A of the annular outlet 108 may allow for an ideal alignment of the sensor-emitter arrangement 220 with the expelled airflow 109. Alternatively or additionally, this may allow the sensor-emitter arrangement 220 to be equidistant from the heater 330 or the heated airflow. This may, in turn, eliminate or reduce the extent to which hotspots develop on the sensor-emitter arrangement 220.

In other examples (not shown) the sensor-emitter arrangement 220 may be placed anywhere radially inwardly of the annular outlet 108. For example, the dashed line R shown in FIG. 3 represents a continuum of locations that are radially inward of the annular outlet 108. Although the dashed line R has a specific rotational position about the axis A, it will be appreciated that the dashed line R could be drawn instead with any rotational position about the axis A, i.e. that in some examples the sensor-emitter arrangement 220 may be located anywhere radially inwardly of the annular outlet 108. In the illustrated example, the sensor-emitter arrangement 220 is set back within the bore 334 from the annular outlet 108, but it will be appreciated that the sensor-emitter arrangement 220 is nonetheless located radially inwardly of the annular outlet 108. Indeed, in other examples (not shown) the sensor-emitter arrangement 220 may be placed at any axial position relative to the annular outlet 108, for example anywhere along the central axis A of the annular outlet 108.

In the illustrated example, the main body 104 comprises an annular heater 330 over which the airflow passes before being expelled, and the sensor-emitter arrangement 220 is located radially inwardly of the annular heater 330. This may allow for the temperatures to which the sensor-emitter arrangement 220 is exposed to be reduced, which may in turn reduce heat protection that may otherwise be needed for the sensor 361, 363 or emitter 361 and/or allow for the sensor 361, 363 or emitter 361 that may be otherwise be damaged by heat or suffer from other interference to nonetheless be used.

In the illustrated example, the sensor-emitter arrangement 220 comprises a capsule 338 housing the at least one of a sensor 361, 363 and an emitter 361. The capsule 338 is connected to the main body 104, and specifically the inner wall 301b of the barrel section 106, by a support member 335. This arrangement may allow for minimal heat to be transferred from main body 104 to the capsule 338. For example, the capsule 338 may be connected to the main body 104 by a single rib 335, which may minimise the heat transferred from the main body 104 to the capsule 338. As illustrated, in some examples the capsule may be elongate in a direction D parallel that of the expelled airflow 109. This may allow to reduce interference of the capsule 338 with a secondary airflow that may be drawn through haircare appliance, specifically through the bore 334 of the barrel section 106 of the main body 104 of the haircare appliance 102, when air is expelled from the annular outlet 108. This may allow for improved operation of the haircare appliance 102.

As mentioned, the haircare appliance 102 comprises a control module 315. In some examples, the control module 315 may be configured to control operation of the haircare appliance 102 in dependence on an output of the sensor-emitted arrangement 220. For example, the sensor-emitter arrangement 220 may be connected by wired or wireless means to the control module 315 and be configured to output data, such as a value of one or more sensing parameters, to the control module 315. For example, the sensor-emitter arrangement may comprise a proximity sensor 361 and the data output may be indicative of a proximity of an object, such as hair or an attachment 114, 116 to the proximity sensor 361. As another example, the sensor-emitter arrangement may comprise a magnetometer 363 and the data output may be indicative of a magnetic field at the magnetometer 363. The control module may be configured to control operation of the haircare appliance 102 in dependence on such output. This may allow for the haircare appliance to be automatically controlled. For example, operation of the haircare appliance may comprise controlling the heater 330 and/or the airflow generator 332 of the haircare appliance 102. For example, where the sensor 361, 363 senses a certain attachment 114, 116, the control module may control the haircare appliance 102 to operate (e.g. the heater 330 and/or the airflow generator 332 to operate) with settings according to the certain attachment 114. For example, the attachments may comprise a diffuser 116 and a concentrator 114. The diffuser 116 may provide better styling results when a relatively low airflow rate is used, whereas the concentrator 114 may provide better styling results when a relatively high airflow rate is used. Accordingly, the control module 315 may be configured to control the rate of the airflow generated by the airflow generator 332 based on the attachment 114, 116 that is attached to the main body 104. As another example, the sensor-emitter arrangement 220 may comprise a sensor 363 configured to sense a property of hair 404 proximate to the haircare appliance, and the control module 315 may be configured to control operation of the haircare appliance 102 based on the sensed property. For example, the sensor-emitter arrangement 220 may comprise a temperature sensor configured to sense a temperature of the user's hair 404, and if the control module 315 determines that a sensed temperature is above a threshold, the control module 315 may control the heater 330 to reduce the extent to which the heater 330 heats the airflow. As another example, the sensor-emitter arrangement 220 may comprise a proximity sensor 361 configured to sense a proximity of hair 404 to the sensor-emitter arrangement 220 (and hence haircare appliance 102), and the control module 315 may be configured to control the heater 330 to increase its temperature and/or control the airflow generator 332 to increase the airflow rate in response to a determination that hair is proximate to the haircare appliance 102. Other properties of hair, such as moisture, may be sensed and operation of the heater 330, airflow generator 332, emitter 361 or other components of the haircare appliance 102 may be controlled by the control module 315 accordingly. As such, the control module 315 may be configured to control operation of the haircare appliance based on a sensed property of hair proximate to the haircare appliance 102.

As mentioned, the sensor-emitter arrangement 220 comprises at least one of a sensor 361, 363 and an emitter 361 configured to sense an attachment 114, 116 attached to the main body 104, or to sense a property of or to irradiate hair proximate the haircare appliance 102. As described in more detail below, examples of sensors 361, 363 configured to sense an attachment 114, 116 attached to the main body 104 include a magnetometer 363 and a Time-of-Flight (TOF) sensor 361. As described in more detail below, an example of a sensor 361 configured to sense a property of hair (not shown) proximate to the haircare appliance 102 includes a proximity sensor such as the TOF sensor 361. As described in more detail below, examples of an emitter 361 configured to irradiate hair proximate the haircare appliance 102 include the TOF sensor 361, as well as a hair, skin or scalp treatment device (not shown) such as a Light Therapy or Phototherapy emitter such a Red Light Therapy emitter, Low-Level Laser Therapy emitter, or an Ultraviolet C (UVC) emitter. A Light Therapy or Phototherapy emitter may emit electromagnetic radiation (i.e. light) in a certain wavelength range. A Red Light Therapy emitter may emit light with wavelengths in the red and/or near infrared (e.g. around 600 nm to around 850 nm). A Low-Level Laser Therapy emitter may be used as a therapy for hair loss and may emit light in the visible to near infrared range (e.g. around 670 nm to around 950 nm). A UVC emitter may be used for sterilisation for example to kill microorganisms and may emit light in the UV C range, (e.g. around 100 nm to around 280 nm). Other examples of sensors or emitters may be used. For example, the sensor may comprise a moisture sensor to sense a moisture of the hair and/or a temperature sensor to sense a temperature of the hair. These may be provided, for example, by a sensor employing spectroscopic techniques such as spectroscopy, imaging, and reflectometry.

In some examples, such as that illustrated, the sensor 361, 363 may be a remote sensor and/or the emitter 361 may be a remote emitter in the sense that the remote sensor 361, 363 and/or remote emitter 361 are configured to sense an attachment 114, 116 attached to the main body 104 or sense a property of or irradiate hair proximate to the haircare appliance 102 remotely (e.g. without contacting) the attachment 114, 116 itself and/or the hair 404 itself. This may provide for flexibility in the placement of the sensor-emitter arrangement 220.

In some examples, the sensor-emitter arrangement 220 may comprise at least one of a light-based sensor 361 and a light-based emitter 361. For example, as mentioned the sensor-emitter arrangement 220 may comprise one or more of a light-based TOF sensor (which may comprise both a light based emitter and a light-based sensor), an imaging device such as a camera, a spectroscopy and/or reflectometry device, and a light-based hair, skin or scalp treatment device such as a Light Therapy or Phototherapy emitter. Being located radially inwardly of the annular outlet 108 may allow for the light emitted from the emitter and/or light received by the sensor to be aligned with the expelled airflow 109, which may allow for more accurate sensing of and/or emitting onto the target of the expelled airflow 109.

The light-based sensor 361 may be configured to sense light in a first direction 440 and/or the light-based emitter may be configured to emit light in the first direction 440, and the first direction 440 may be parallel to the central axis A of the annular outlet 108. This may help ensure that the emitted and/or received light is aligned with the expelled airflow 109, which may allow for more accurate sensing of and/or emitting onto the target of the expelled airflow 109. In the example illustrated in the Figures, the first direction 440 may be aligned or co-linear with the central axis A of the annular outlet 108. This may further ensure that the emitted and/or received light is aligned with the expelled airflow 109.

As mentioned, the sensor-emitter arrangement 110 may comprise a proximity sensor 361 such as the TOF sensor 361 and/or a magnetometer 363. Locating the proximity sensor 361 radially inwardly of the annular outlet 108 may allow for an alignment of the target of the proximity sensor (such as a user's head or hair) with the airflow target (i.e. the user's head or hair). This may, in turn, allow for an accurate and/or reliable determination of a proximity, e.g. a distance to a user's head or hair, and hence e.g. allow for improved operation of the haircare appliance 102. As described in more detail below, the magnetometer 363 may allow for magnetic attachments 114, 116 to be discriminated between without providing a magnetometer at an attachment interface 339 of the main body 104 and the attachment 114, 116, which may have tight packaging constraints. Alternatively or additionally, placing the magnetometer 363 radially inwardly of the annular outlet 108 may reduce interference from other electrical components of the haircare appliance 102 such as the heater 330.

As mentioned, in the illustrated example, the haircare appliance 102 comprises the barrel section 106 that comprises the annular outlet 108, the barrel section 106 comprises the bore 334, and the sensor-emitter arrangement 220 is located in the bore 334. Locating the sensor-emitter arrangement 220 in the bore 334 may allow for the sensor-emitter arrangement 220 to be relatively physically isolated from components located in the annular barrel section 106, such as the heater 330, which may reduce heat transfer and/or other interference from those components. In the illustrated example, the bore 334 is an open-ended bore 334 in that the bore 334 extends all the way through the barrel section 106 (is not a blind bore) and the ends of the bore 334 are not covered. Locating the sensor-emitter arrangement 220 in the bore 334 may therefore allow a clear line-of-sight from the sensor-emitter arrangement 220 out of the barrel section 106 of the haircare appliance 102. This may, for example, be useful where the sensor 361 senses an attachment 114, 116 or a property of hair using a light-based sensing method and/or where the emitter 361 irradiates hair proximate to the haircare appliance 102.

Further, in the illustrated example, the main body 104, and specifically the barrel section 106 of the main body 104, surrounds or houses the sensor-emitter arrangement 220. This may allow for the main body 104 to physically protect the sensor-emitter arrangement 202, which may provide for a robust haircare appliance.

In the illustrated example, sensor-emitter arrangement 220 comprises a visual indicator device 367 configured to indicate a current operating mode of the haircare appliance 102 to a user. For example, the visual indicator device 367 may be an LED. In the illustrated example, visual indicator device 367 is configured to provide a visual indication in a direction opposite the direction D of the expelled airflow 109. This may allow a user to view the visual indication device 367 without the expelled airflow 109 being directed at the user. In some examples, the visual indicator device 367 may indicate an operating mode of the sensor-emitter arrangement 220, for example whether the sensor-emitter 220 is currently operating or which sensing or emitting mode the sensor-emitter arrangement 220 is currently operating in. The sensor-emitter arrangement 220 comprising the visual indicator device 367 may allow for an operating mode to be communicated to the user without significantly altering a display configuration of the main body 104 of the haircare appliance 102 itself, which may be cost effective. In some examples, the sensor-emitter arrangement 220 may comprise other sensors and/or emitters, such as an accelerometer (not shown) configured to sense changes in direction of movement of the haircare appliance 102.

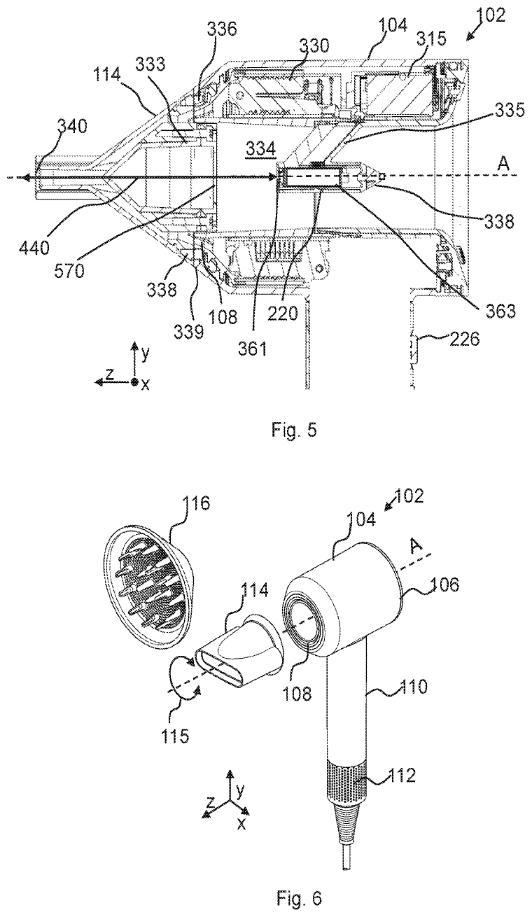

As best seen in FIGS. 5 and 6, in some examples the haircare appliance 102 may comprise an attachment 114, 116 attachable to the main body 104 to receive the expelled airflow 109 from the annular outlet 108. In these cases, the sensor-emitter arrangement 220 may be configured to sense the attachment 114, 116, or to sense a property of or to irradiate hair proximate to the attachment 114, 116, when the attachment is attached to the main body 104. This may allow for the sensor-emitter arrangement 220 to function in the case where an attachment 114, 116 is attached to the main body 104. This may improve the flexibility with which the haircare appliance 102 can be used.

Each of the attachments 114, 116 may be attached to an end of the barrel section 106 of the main unit 104. When attached, each of the attachments 114, 116 may be free to rotate 115 relative to the main unit 104 about the central longitudinal axis A of the barrel section 106 (which in the illustrated example is colinear with the central axis A of the annular outlet 108). The free rotation of the attachments 114, 116 has the advantage that a user is able to achieve a desired direction and angle of airflow without having to hold or manipulate the haircare appliance 102 at uncomfortable angles. In this example, each of the attachments 114, 116 comprises an annular magnet 338, and the barrel section comprises a ferrous ring 336 to which the magnet 338 is attracted to secure the attachment 114, 116 in place. It will be appreciated that the ring 336 need not necessarily be ferrous and may be made of another material to which the magnet 338 is magnetically attracted. It will also be appreciated that the attachment of the attachment 114, 116 to the main body 104 need not necessarily be via magnetic means and that other means of attachment may be used. In the illustrated example, when the attachment 114, 116 is attached to the main unit 104, a portion or bung 333 of the attachment 114, 116 is received into the bore 334. In this example, when the attachment 114, 116 is attached to the main body 104, the airflow in the form of the annular column 109 is expelled from the annular outlet 108 flows through the attachment and is in turn expelled from an outlet 340 of the attachment 114.

The attachment 114, 116 may comprise a transport path along which emissions 440 pass to or from the sensor-emitter arrangement 220. This may allow for emissions 440, such as electromagnetic radiation, to pass from the sensor-emitter arrangement 220 to the head or hair of the user (or vice versa) when the attachment 114, 116 is attached to the main body 104. This may allow for flexibility in the functioning of the haircare appliance (i.e. by allowing use of attachments 114, 116) whilst still allowing for the hair sensing and/or irradiating functionality of the haircare appliance 102. In the illustrated example, the transport path is linear and comprises one or more optical windows 570 in the attachment 114, 116. Each optical window 570 may comprise a hole or a transparent member. In other examples, the transport path through the attachment 114, 116 may be non-linear and may comprise one or more optical elements (e.g. mirrors, lenses, and/or optical fibres) for redirecting the path along which emissions pass to or from the sensor-emitter arrangement 220. The emissions 440 which pass to or from the sensor-emitter arrangement 220 may comprise, for example, one or more of: emissions emitted from an emitter of the sensor-emitter arrangement 220 towards hair, such emissions reflected from hair back towards the sensor-emitter arrangement 220, and emissions emitted by the hair towards the sensor-emitter arrangement 220 due to thermal electromagnetic radiation or fluorescence.

As mentioned, the sensor-emitter arrangement may comprise a time-of-flight (TOF) sensor 361, and the attachment 114, 116 may be one of a plurality of attachments 114, 116. In this case, the control module 315 may be configured to determine which of the plurality of magnetic attachments 114, 116 is attached to the main body 104 based on data output by the time-of-flight sensor 361. This may allow the control module 315 to determine which of the plurality of attachments 114, 116 is attached to the main body. The TOF sensor 361 being located radially inwardly of the annular outlet 108 may allow for the attachment 114, 116 to be determined remotely from the attachment interface 339, which may have tight packaging constraints. Alternatively or additionally, the TOF sensor 361 may be used both to determine which attachment 114, 116 is attached to the main unit 104 and to sense a proximity of hair to the attachment 114, 116. Using one sensor for two functions may provide a cost-effective arrangement, for example as compared to providing separate sensors for the separate functions.

For example, the TOF sensor 361 may be provided as an integrated package or all-in-one system that comprises an emitter, a receiver (i.e. a sensor), and a processor. The emitter emits emissions, which in this example are photons of electromagnetic radiation. The emitter emits the emissions as discrete pulses, with a frequency typically of the order of MHz. In other examples, the emissions may comprise acoustic (e.g., ultrasonic) pulses. The sensor then receives reflected emissions, which have been reflected and returned to the TOF sensor 361. The processor then determines time differences between emitting and receiving the emissions and from this calculates a distance between the TOF sensor 361 and a target responsible for the reflected emissions. The processor then outputs this distance data to the control module 315. The TOF sensor 361 may be used to sense which, if any, of the attachments 114, 116 is attached to the main unit 104 and/or may be used to sense the proximity of a user's head or other object to the haircare appliance 102. For example, the control module 315 may analyse the distance data output by the TOF sensor 361 and from this analysis determine (i) which attachment, if any, is attached to the main body 104 and (ii) the proximity of the head of a user.

For example, each of the attachments 114, 116 may comprise a projection 333 that projects into the bore 334 of the barrel section 106 towards the TOF sensor 361. The emissions emitted by the TOF sensor 361 reflect off the projection 333 and are returned to the TOF sensor 361. The projections 333 of the attachments 114, 116 may differ such that the emissions reflected by the projection of each attachment differ, e.g., have a different signature. For example, the projections 333 differ in size. For example, lengths of the projections 333 may differ such that, when a particular attachment 114, 116 is attached to the main body 104, the projection 333 projects towards the TOF sensor 361 by a different amount to those of other attachments 114, 116. The distance between the TOF sensor 361 and the projection 333 therefore differs for different attachments 114, 116. This is reflected in the data output by the TOF sensor 361, which the control module 315 may use to determine which of the attachments 114, 116, if any, is attached to the main body 114. In other examples, the projections 333 may differ in additional or alternative ways, such as shape, surface features, or indeed any other feature that changes the way in which emissions are reflected by the projection 333.

As mentioned, each of the attachments 114, 116 is attachable to the main body 114 in any one of a plurality of rotational positions 115 about a rotational axis A (which in the illustrated examples is the same as the central longitudinal axis A of the barrel section and the central axis A of the annular outlet 108). Locating the sensor-emitter arrangement 220 on the rotational axis A allows for the TOF sensor 361 to receives the same reflected emissions from an attachment 114, 116 irrespective of the rotational position of the attachment 114, 116. This may, in turn, allow the TOF sensor 361 to sense which of the attachments 114, 116 is attached to the main body 104 irrespective of the rotational position of the attachment 114, 116. Alternatively or additionally, this may allow for the emissions 340 to travel to and/or from the TOF sensor 361 through the transport path of the attachment 114, 116 to and/or from the head of the user in the same way regardless of the rotational position of the attachment 114, 116. Accordingly, the TOF sensor 361 may sense a distance to a user's head or other object irrespective of the rotational position of the attachment 114, 116. This may provide for flexible use of the attachment 114, 116 while still allowing the attachment sensing and/or head distance sensing functionality to be provided for.

As mentioned, in some examples, the sensor-emitter arrangement 220 may comprise a magnetometer 363 and the attachment 114, 116 may be one of a plurality of magnetic attachments 114, 116. In these examples, the control module 315 may be configured to determine which of the plurality of magnetic attachments 114, 116 is attached to the main body 104 based on data output by the magnetometer 363. This may allow for the control module 315 to determine which of a plurality of magnetic attachments 114, 116 is attached to the main body. For example, the different magnetic attachments 114, 116 may differ in the magnetic field produced, at the magnetometer 363, by the magnet 338 of the attachment 114, 116 when the magnetic attachment 114, 116 is attached to the main body 104. This may be sensed by the magnetometer 363, and the control module 315 may identify a given one of the magnetic attachments 114, 116 from among the plurality based on the output from the magnetometer 363 indicating a given magnetic field at the magnetometer 363. For example, the differing magnetic field may be produced by differing arrangements of one or more magnetic elements, for example by arrangements of magnetic elements differing in size, magnetic strength, physical distribution, magnetic polarity, and/or magnet orientation. The magnetometer 363 being located radially inwardly of the annular outlet 108 may allow for the attachment 114, 116 to be determined remotely from the attachment interface 339 (i.e. the region of the main body 104 where attachments 114, 116 attach to the main body 104), which may have tight packaging constraints.

As mentioned, each of the attachments 114, 116 is attachable to the main body 114 in any one of a plurality of rotational positions 115 about a rotational axis A (which in the illustrated example is the same as the central longitudinal axis A of the barrel section 106 and the central axis A of the annular outlet 108). Locating the sensor-emitter arrangement 220 on the rotational axis A may allow for the magnetometer to experience the same magnetic field (at least the same component of the magnetic field at the magnetometer 363 in a direction parallel to rotational axis A) produced by a given magnetic attachment 114, 116, irrespective of the rotational position of the attachment 114, 116. This may, in turn, allow the magnetometer 363 to sense which of the attachments 114, 116 is attached to the main body 104 irrespective of the rotational position of the attachment 114, 116.

Indeed, in some examples, the sensor-emitter arrangement 220 may be located on the central axis A of the annular outlet 108 and the attachment 114, 116, when attached to the main body 104, may be rotatable relative to the main body about the central axis A. This may allow that the sensor-emitter arrangement 220 is aligned with a rotational axis of the attachment 114, 116. This may allow the sensor-emitter arrangement 220 to function regardless of the rotational position of the attachment 114, 116. This may improve flexibility of use of the attachment 114, 116. For example, this may allow that the sensor-emitter arrangement 220 may be aligned with the emissions transport path of the attachment 114, 116 regardless of the rotational position of the attachment and/or that a TOF sensor 361 or a magnetometer 220 of the sensor-emitter arrangement 220 can identify an attachment 114, 116 regardless of the rotational position of the attachment 114, 116. The attachment being rotatable relative to the main body about the central axis may allow for improved functionality and/or ease of use of the haircare appliance 102. Other configurations are possible.

Although in the examples described above with reference to FIGS. 1 to 6 the haircare appliance 102 was described as having a certain form and having certain components and features, it will be appreciated that this need not necessarily be the case, and that according to examples there may be provided any haircare appliance comprising a main body comprising an annular outlet configured to expel an airflow in the form of an annular column; and a sensor-emitter arrangement located radially inwardly of the annular outlet, the sensor-emitter arrangement comprising at least one of a sensor and an emitter configured to sense an attachment attached to the main body, or to sense a property of or to irradiate hair proximate the haircare appliance. It will also be appreciated that this haircare appliance may have any one or combination of the components and/or features of the example haircare appliance 102 described above with reference to FIGS. 1 to 6.

Further, in another aspect, there may be provided a haircare appliance comprising a main body comprising a barrel section having a central bore and an outlet located at an end of the barrel section to expel an airflow in the form of an annular column; and a sensor-emitter arrangement located within the bore, the sensor-emitter arrangement comprising at least one of a sensor and an emitter. In particular it is noted that in examples of this other aspect the sensor may be any sensor and/or the emitter may be any emitter. In some examples of this other aspect, the haircare appliance may have components and/or features that are the same or similar to those of the haircare appliance 102 described above with reference to FIGS. 1 to 6. For example, the haircare appliance, the main body, the barrel section, the central bore, the outlet, the expelled airflow in an annular column, the sensor-emitter arrangement, the sensor and/or the emitter of this other aspect may be the same or have the same or similar functionality to the haircare appliance 102, the main body 104, the barrel section 106, the central bore 334, the outlet 108, the expelled airflow in an annular column 109, the sensor-emitter arrangement 220, the sensor 361, 363 and/or the emitter 361, respectively, described above with reference to FIGS. 1 to 6. In some examples of this other aspect, the haircare appliance may have any one or combination of the components and/or features of the example haircare appliance 102 described above with reference to FIGS. 1 to 6. In any case, the sensor-emitter arrangement 220 being located within the bore 334 may allow the sensor-emitter arrangement 220 to be aligned with the expelled airflow 109 path or a target of the expelled airflow path (such as a user's head or hair) without disrupting the expelled airflow 109 itself. Accordingly, accurate sensing and/or emitting may be provided without negatively affecting or impacting the expelled airflow 109. Alternatively or additionally, locating the sensor-emitter arrangement 220 within the bore 334 may allow for the sensor-emitter arrangement 220 to be relatively physically isolated from other components located in the barrel section 106, such as a heater 330, which may, in turn, reduce heat transfer and/or other interference from those components. Alternatively or additionally, locating the sensor-emitter arrangement 220 within the bore 334 may allow the sensor 361, 363 or emitter 363 to be incorporated into the haircare appliance 102 without requiring significant modification of the packaging of the haircare appliance 102 and/or increasing the overall size of the hair care appliance 102, which may be cost effective. Alternatively or additionally, locating the sensor-emitter arrangement 220 within the bore 334 may allow a clear line-of-sight from the sensor-emitter arrangement 220 out of the haircare appliance 102. This may, for example, be useful where the sensor 361 senses an attachment 114, 116 or a property of hair using a light-based sensing method and/or where the emitter 361 irradiates hair proximate to the haircare appliance.

Whilst particular examples have been described, it should be understood that these are illustrative examples only and that various modifications may be made without departing from the scope of the invention as defined by the claims.

1. A haircare appliance comprising:
a main body comprising an annular outlet configured to expel an airflow in the form of an annular column; and
a sensor-emitter arrangement located radially inwardly of the annular outlet, wherein the sensor-emitter arrangement comprises at least one of a sensor and an emitter configured to sense an attachment attached to the main body, or to sense a property of or to irradiate hair proximate the haircare appliance.

2. The haircare appliance according to claim 1, wherein the haircare appliance comprises a barrel section that comprises the annular outlet, the barrel section comprises a bore, and the sensor-emitter arrangement is located in the bore.

3. The haircare appliance according to claim 2, wherein the main body surrounds or houses the sensor-emitter arrangement.

4. The haircare appliance according to claim 1, wherein the sensor-emitter arrangement is located on a central axis of the annular outlet.

5. The haircare appliance according to claim 1, wherein the sensor-emitter arrangement comprises at least one of a light-based sensor and a light-based emitter.

6. The haircare appliance according to claim 5, wherein the light-based sensor is configured to sense light in a first direction or the light-based emitter is configured to emit light in the first direction, and the first direction is parallel to the central axis of the annular outlet.

7. The haircare appliance according to claim 1, wherein the sensor-emitter arrangement comprises a proximity sensor or a magnetometer.

8. The haircare appliance according to claim 1, wherein the sensor-emitter arrangement comprises a visual indicator device configured to indicate a current operating mode of the haircare appliance to a user.

9. The haircare appliance according to claim 1, wherein the main body comprises an annular heater over which the airflow passes before being expelled, and the sensor-emitter arrangement is located radially inwardly of the annular heater.

10. The haircare appliance according to claim 1, wherein the sensor-emitter arrangement comprises a capsule housing the at least one of a sensor and an emitter, and the capsule is connected to the main body by a support member.

11. The haircare appliance according to claim 10, wherein the capsule is elongate in a direction parallel that of the expelled airflow.

12. The haircare appliance according to claim 1, further comprising a control module configured to control operation of the haircare appliance in dependence on an output of the sensor-emitted arrangement.

13. The haircare appliance according to claim 1, further comprising an attachment attachable to the main body to receive the expelled airflow from the annular outlet, and the sensor-emitter arrangement is configured to sense the attachment, or to sense a property of or to irradiate hair proximate to the attachment, when the attachment is attached to the main body.

14. The haircare appliance according to claim 13, wherein the attachment comprises a transport path along which emissions pass to or from the sensor-emitter arrangement.

15. The haircare appliance according to claim 13, wherein the sensor-emitter arrangement comprises a magnetometer, the attachment is one of a plurality of magnetic attachments, and the haircare appliance comprises a control module configured to determine which of the plurality of magnetic attachments is attached to the main body based on data output by the magnetometer.

16. The haircare appliance according to claim 1, wherein the sensor-emitter arrangement comprises a time-of-flight sensor, the attachment is one of a plurality of attachments, and wherein the haircare appliance comprises a control module configured to determine which of the plurality of magnetic attachments is attached to the main body based on data output by the time-of-flight sensor.

17. The haircare appliance according to claim 1, wherein the sensor-emitter arrangement is located on a or the central axis of the annular outlet and the attachment, when attached to the main body, is rotatable relative to the main body about the central axis.

18. A haircare appliance comprising:
a main body comprising a barrel section having a central bore and an outlet located at an end of the barrel section to expel an airflow in the form of an annular column; and
a sensor-emitter arrangement located within the bore, the sensor-emitter arrangement comprising at least one of a sensor and an emitter.
